(19) United States
(12) Patent Application Publication
(10) Pub. No.: US 2014/0262733 A1
(43) Pub. Date: Sep. 18, 2014

(54) CAUSTIC TREATMENT OF FORMALDEHYDE RECYCLE COLUMN FEED

(71) Applicants: Hashim M. Badat, Houston, TX (US); Jason C. Gause, Baytown, TX (US)

(72) Inventors: Hashim M. Badat, Houston, TX (US); Jason C. Gause, Baytown, TX (US)

(21) Appl. No.: 14/360,177

(22) PCT Filed: Sep. 13, 2012
(86) PCT No.: PCT/US2012/055149

§ 371 Date: May 22, 2014

Related U.S. Application Data
(60) Provisional application No. 61/563,875, filed on Nov. 28, 2011.

(51) Int. Cl. C07C 29/80

(52) U.S. Cl. CPC C07C 29/80
USPC 203/6

(57) ABSTRACT

An improved method for removing formaldehyde from a crude butynediol product stream comprising the step of providing a crude butynediol stream containing butynediol and formaldehyde. A pH control agent is then mixed with the crude butynediol stream to form a treated product stream, wherein the pH of the treated product stream is raised to a level that limits the reaction of butynediol and formaldehyde to form acetal complex and decrease the solubility of trace metals. The treated product stream then flows into the inlet of a continuous distillation column. Finally, a concentrated formaldehyde stream from the overhead stream of the distillation column and a concentrated butynediol stream from the distillation bottoms stream that is essentially free of formaldehyde and acetal complex are both recovered.

### CROSS-REFERENCE TO RELATED APPLICATION

This application claims the benefit of priority of U.S. Provisional Application 61/563,875 filed Nov. 28, 2011, the disclosure of which is incorporated herein by reference in its entirety.

### FIELD OF THE INVENTION

This disclosure relates to a process for producing 1,4 butynediol. More specifically, it relates the improved efficiency during the distillation recovery of excess formaldehyde from crude 1,4 butynediol solutions.

### BACKGROUND OF THE INVENTION

1,4-Butynediol (BYD) is a commonly produced organic compound. BYD is a useful intermediate chemical in the production of pesticides, textile additives, corrosion inhibitors, plasticizers, synthetic resins, butanediol, tetrahydrofuran, polyether polyols and polyurethanes.

It is well known that BYD can be produced by the Reppe process, via the reaction of acetylene and formaldehyde. Several patents, herein incorporated by reference, such as U.S. Pat. Nos. 2,300,969, 3,560,576, 4,093,668 and 4,127,734, teach the production of BYD through Reppe chemistry.

Many commercial processes for the synthesis of BYD operate with excess formaldehyde, thereby improving yields. In the process of manufacturing BYD, an aqueous solution, which is referred to as crude BYD, may contain 30 to 40% BYD and 3 to 12% formaldehyde. Once a crude BYD stream has been produced, it is desirable to remove unreacted formaldehyde from the stream to retrieve a purified BYD product stream that can be further processed. Generally, the crude BYD can be distilled to remove formaldehyde which is recycled in the system. The distillation step is expensive both in the initial cost of equipment and in energy consumption during use.

Several other known process also exist for removing excess formaldehyde from a BYD stream. For example, European Pat No. 309915 teaches a process wherein excess formaldehyde is removed from aqueous solutions of BYD by adding methanol and an acidic agent to the aqueous BYD solution and distilling off the dimethyl formal from the mixture at elevated temperatures. U.S. Pat. No. 5,973,213 teaches the separation of solids from aqueous BYD solutions by passing a solids-containing aqueous BYD solution in the downflow mode through a column and thus bringing it into contact with a solvent which has a lower density than the solids-containing BYD solution and forms a second phase with the latter, with the solvent rising in countercurrent to the aqueous BYD solution, the solid accumulating at the interface between the aqueous BYD and the solvent and the solid being removed from the column by taking off a mixture of aqueous BYD and solvent.

U.S. Pat. No. 7,605,292 relates to a process that comprises compressing BYD to from 50 to 1500 bar, depressurizing it, waiting for phase separation to occur after depressurization and separating off the bottom phase. U.S. Pat. No. 4,180,687 teaches the removal of formaldehyde from crude BYD, wherein, the formaldehyde can be reacted to a polymeric substance in the presence of NaOH or Na.sub.2 CO.sub.3 at high temperatures. U.S. Pat. No. 4,319,055 relates to a process for removing formaldehyde from aqueous solutions of BYD by treating the solutions with an alkaline agent at an elevated temperature in the presence of hydrogen peroxide. All of these processes involve costly intermediate steps and additional processing equipment in order to remove excess formaldehyde from the crude BYD stream. Therefore, it is desirable to improve the existing distillation process for purifying BYD, so that the column efficiency is increased and the energy required for the process is decreased.

It is readily apparent from the physical properties of the major components of crude BYD that distillation would lend itself to separating the excess CH₂O from the crude BYD for recycle to the first reaction step. In a formaldehyde recycle distillation column, the formaldehyde is removed so that formaldehyde and some of the water is distilled overhead and recycled directly or indirectly back to the first reaction step. The bottoms from the distillation column, is comprised of a concentrated BYD stream and is fed forward for further processing.

It has been found that under typical operating conditions, the column operates at a lower effective capacity than would be predicted based upon the previously understood behavior of the components to be separated. Additionally, the column requires a higher steam duty than would normally be expected. It has not been fully understood why the operation appears to operate inefficiently.

Therefore, there is a need for a method of improving the recycle distillation column efficiency and decreasing the energy usage in a process for removing formaldehyde from a crude BYD stream.

### SUMMARY OF THE INVENTION

The present invention relates to a process for improving the recycle distillation column efficiency and decreasing the energy usage in a process for removing formaldehyde from a crude 1,4 butynediol (BYD) stream by controlling the pH level of the crude BYD stream prior to removing the formaldehyde.

The crude BYD stream is produced from a process of forming BYD through the reaction of formaldehyde and acetylene. An embodiment of the present invention comprises the steps of:
(a) providing a crude butynediol stream containing butynediol and formaldehyde;
(b) increasing the pH of the crude butynediol stream and forming a treated product stream;
(c) flowing said treated product stream into the inlet of a continuous distillation column;
(d) recovering a concentrated formaldehyde stream from the overhead stream of the distillation column; and
(e) recovering a concentrated butynediol stream from the bottoms stream of the distillation column.

In another embodiment, the pH of the treated product stream is raised to a level that limits the reaction of butynediol and formaldehyde to form acetal complex and decreases the solubility of trace metals found in the treated product stream, wherein the trace metals precipitate in the treated product stream.

In another embodiment, the pH level of the treated product stream is increased to a range of 5.5 to 10.

In another embodiment, the pH level of the treated product stream is increased through the addition of a pH control agent.

In another embodiment, the pH control agent is an aqueous sodium hydroxide solution.

In another embodiment, the pH of the treated product stream is increased though the use of ion exchange resins.

In another embodiment, the concentration of formaldehyde in the distillation bottoms stream is 1.0% by weight or less.

In another embodiment, the concentration of formaldehyde in the distillation bottoms stream is from 0.1 to 1.0% by weight.

In another embodiment, the process further comprises the step of flowing said treated product stream through a filtration system to remove trace metals prior to step (c).

Another embodiment of the present invention discloses a method for reducing the steam consumption needed for a process for removing formaldehyde from a crude butynediol product stream comprising the steps of:
(a) providing a crude butynediol stream containing butynediol and formaldehyde;
(b) increasing the pH of the crude butynediol stream and forming a treated product stream;
(c) flowing said treated product stream into the inlet of a continuous distillation column;
(d) recovering a concentrated formaldehyde stream from the overhead stream of the distillation column; and
(e) recovering a concentrated butynediol stream the bottoms stream of the distillation column.

In another embodiment, the pH of the treated product stream is raised to a level that limits the reaction of butynediol and formaldehyde to form acetal complex and decreases the solubility of trace metals found in the treated product stream, wherein the trace metals precipitate in the treated product stream.

In another embodiment, the pH level of the treated product stream is increased to a range of 5.5 to 10.

In another embodiment, the pH level of the treated product stream is increased through the addition of a pH control agent.

In another embodiment, the pH control agent is an aqueous sodium hydroxide solution.

In another embodiment, the pH of the treated product stream is increased though the use of ion exchange resins.

In another embodiment, the concentration of formaldehyde in the distillation bottoms stream is 1.0% by weight or less.

In another embodiment, the concentration of formaldehyde in the distillation bottoms stream is from 0.1 to 1.0% by weight.

In another embodiment, the method further comprises the step of flowing said treated product stream through a filtration system to remove trace metals prior to step (c).

### BRIEF DESCRIPTION OF THE DRAWINGS

FIG. 1 is a process diagram for an embodiment of the present invention.

FIG. 2 is a chart showing the steam consumption of the recycle distillation column as the pH on the column inlet stream is increased.

### DETAILED DESCRIPTION OF THE INVENTION

The present invention relates to a process for improving the recycle distillation column efficiency and decreasing the energy usage in a process for removing formaldehyde from a crude 1,4 butynediol (BYD) stream by controlling the pH level of the crude BYD stream prior to removing the formaldehyde.

All patents, patent applications, test procedures, priority documents, articles, publications, manuals, and other documents cited herein are fully incorporated by reference to the extent such disclosure is not inconsistent with this invention and for all jurisdictions in which such incorporation is permitted.

The term “BYD”, as used herein, unless otherwise indicated, refers to 1,4 butynediol. The general formula for BYD is HOCH₂CCCH₂OH. BYD is also known as butynediol, but-2-yne-1,4,diol, 2-Butyne-1,4-diol and 1,4-dihydroxy-2-butyne.

The term “acetal complex”, as used herein, unless otherwise indicated, refers to acetal and hemiacetal compounds that are formed from the reaction of BYD and formaldehyde in the distillation column. The general formula for an acetal is R¹HC(OR)²OR³, where R¹, R², or R³ are often hydrogen. The general formula for a hemiacetal is R¹R²C(OH)OR, where R¹ or R² is often hydrogen.

The term “caustic addition”, as used herein, unless otherwise indicates, refers to a strong basic solution. In specific embodiments of the present invention, sodium hydroxide (NaOH) may be utilized as a caustic addition.

Referring to FIG. 1, an exemplary embodiment of the present invention is herein described. Crude BYD is produced via the reaction of acetylene and formaldehyde (CH₂O) (100). It is desirable to remove excess formaldehyde from the crude BYD stream (120), before the BYD is further processed. This can be accomplished in recycle distillation column (200), wherein a purified BYD product stream (220), is recovered from the recycle distillation column bottoms and the excess formaldehyde (240, 260) is recovered from the top of the column and can be recycled to further produce BYD.

In normal operation, the BYD would go down the column because of its relatively high boiling point, along with some water. CH₂O would tend to go up the column, with some water. In one embodiment, the CH₂O ranges from about 5 to 12% at the feed point, down to 0.1 to 1.0% in the bottoms from the column (220). The concentration of CH₂O going up the column ranges from 5 to 12% at the feed point, to 20 to 30% in the overheads (240, 260).

The applicants have discovered that a caustic addition (140) to the crude BYD stream (120) to form a treated product stream (160) to the feed to the recycle distillation column (200) improves the efficiency in separating the CH₂O and water overhead (240, 260) for recycle and significantly reduces steam use by up to 25% of the steam that would otherwise be consumed. The underlying mechanism is not fully understood, and while not to limit the disclosed process by a recitation of theory, it is believed that increasing the pH in accordance with the disclosed process inhibits the formation of constituents that otherwise interfere with efficient operation of the recycle distillation column (200). One theory is that the reduction in steam use arises because of a reduction in acetal complex, which comprises of acetals and hemiacetals made from the reaction of BYD and CH₂O. The caustic addition (140) lowers the pH of the crude BYD stream (120) by neutralizing the organic acids in the stream to form organic salts and water. For example, formic acid that maybe present in the crude BYD stream (120) will be neutralized to sodium formate by the addition of a (caustic) sodium hydroxide addition.

CH₂O in aqueous solutions reacts with water in an equilibrium fashion to make methanol (MeOH) and formic acid HCOOH) as by-products as a function of time. Given time, the pH of the crude BYD will increase in HCOOH content thereby reducing the pH of the crude BYD to between 4 and 4.5. Caustic is added to raise the pH to a range of about 5.5 to about 10 prior to feeding the crude BYD to the CH₂O recycle column. In an embodiment of the present invention, the caustic treatment may be an aqueous sodium hydroxide solution.

In other embodiments of the current invention, the pH of the crude BYD stream (120) maybe increased through other methods known in the art. In a particular embodiment of the current invention, the pH of the crude BYD stream (120) can be increased by the removal of organic acids through the use of ion exchange resins. An example of ion exchange resins that may be used are anion exchange resins such as AMBERLITE™ IRA96 or DOWEX™ M-43 that act as acid absorbers to neutralize the crude BYD stream (120). Other known methods of removing organic acids from process stream may also be used. For example, an adsorbent such as activated carbon may also be used to neutralize the crude BYD stream (120).

The applicants have also found that increasing the pH of the treated product stream (160) significantly decreases the solubility of trace metals, such as copper, that are found in the crude BYD stream (120). Once the solubility of the trace metals has been decreased, the precipitated trace metals may then be removed from the treated product stream though a filtration system (280) prior to entering the recycle distillation column (200). Any filtration system known in the art, such as a cartridge filter system, may be used to remove the trace metals from the treated product stream (160). The removal of these trace metals is beneficial because they can contribute to fouling in the recycle distillation column (200) and also contribute to catalyst poisoning downstream when the purified BYD product stream (220) is further processed.

### EXAMPLES

The following Examples demonstrate the present invention and its capability for use. The invention is capable of other and different embodiments, and its several details are capable of modifications in various apparent respects, without departing from the scope and spirit of the present invention. Accordingly, the Examples are to be regarded as illustrative in nature and non-limiting.

### Example 1

FIG. 2 is a chart showing the observed steam usage at the INVISTA LaPorte site with and without a caustic addition to the recycle distillation column feed. The recycle distillation column (200) was operated at a pressure range between 55-65 psig. The temperature at the feed point (160) was between 50-70° C., the bottoms temperature (220) was between 155-177° C. and the temperature at the top of the column (240) was between 140-150° C. The reflux temperature was between 80-120° C. and the reflux ratio was maintained at 0.5-0.8 (reflux to feed). The chart in FIG. 2 shows the steam consumption data for a recycle distillation column. The pounds of steam used per pound of crude BYD feed Steam were tracked. The data was taken for period when the pH of the BYD inlet stream was unregulated and for a period when the pH was increased by the addition of aqueous sodium hydroxide (25-50% wt NaOH). Prior to the caustic addition, the average pH of the BYD inlet stream was observed to be about 5.35. Over an 18 week period, the pH of the BYD inlet stream was increased and regulated at a range of 5.7-6.4 through the caustic addition. The results of the study are summarized in FIG. 2. The amount of steam used for the column was reduced from 0.95 lb steam/lb feed to 0.72 lb steam/lb feed. This indicates that the efficiency of the column was improved by raising the pH level of the BYD inlet stream to the recycle distillation column.

### Example 2

An improved method for removing formaldehyde from a crude butynediol product stream comprising the step of providing a crude butynediol stream containing butynediol and formaldehyde. The pH of the crude butynediol stream is then increased to form a treated product stream. The treated product stream then flows into the inlet of a continuous distillation column. Finally, a concentrated formaldehyde stream from the overhead stream of the distillation column and a concentrated butynediol stream from the bottoms stream of the distillation column are both recovered.

### Example 3

The process of Example 2 is repeated with additional steps. In this example, the pH of the treated product stream is raised to a level that limits the reaction of butynediol and formaldehyde to form acetal complex and decreases the solubility of trace metals found in the treated product stream, wherein the trace metals precipitate in the treated product stream.

### Example 4

The process of Example 3 is repeated with additional steps. In this example, the pH level of the treated product stream is increased to a range of 5.5 to 10.

### Example 5

The process of Example 4 is repeated with additional steps. In this example, pH level of the treated product stream is increased through the addition of a pH control agent.

### Example 6

The process of Example 5 is repeated with additional steps. In this example, the pH control agent is an aqueous sodium hydroxide solution.

### Example 7

The process of Example 6 is repeated with additional steps. In this example, the pH of the treated product stream is increased though the use of ion exchange resins.

### Example 8

The process of Example 7 is repeated with additional steps. In this example, the concentration of formaldehyde in the distillation bottoms stream is 1.0% by weight or less.

### Example 9

The process of Example 8 is repeated with additional steps. In this example, the concentration of formaldehyde in the distillation bottoms stream is from 0.1 to 1.0% by weight.

### Example 10

The process of Example 9 is repeated with additional steps. In this example, said treated product stream flows through a filtration system to remove trace metals prior to entering the continuous distillation column.

### Example 11

A method for reducing the steam consumption needed for removing formaldehyde from a crude butynediol product stream comprising the step of providing a crude butynediol stream containing butynediol and formaldehyde. The pH of the crude butynediol stream is then increased to form a treated product stream. The treated product stream then flows into the inlet of a continuous distillation column. Finally, a concentrated formaldehyde stream from the overhead stream of the distillation column and a concentrated butynediol stream from the bottoms stream of the distillation column are both recovered.

### Example 12

The process of Example 11 is repeated with additional steps. In this example, the pH of the treated product stream is raised to a level that limits the reaction of butynediol and formaldehyde to form acetal complex and decreases the solubility of trace metals found in the treated product stream, wherein the trace metals precipitate in the treated product stream.

### Example 13

The process of Example 12 is repeated with additional steps. In this example, the pH level of the treated product stream is increased to a range of 5.5 to 10.

### Example 14

The process of Example 13 is repeated with additional steps. In this example, pH level of the treated product stream is increased through the addition of a pH control agent.

### Example 15

The process of Example 14 is repeated with additional steps. In this example, the pH control agent is an aqueous sodium hydroxide solution.

### Example 16

The process of Example 15 is repeated with additional steps. In this example, the pH of the treated product stream is increased though the use of ion exchange resins.

### Example 17

The process of Example 16 is repeated with additional steps. In this example, the concentration of formaldehyde in the distillation bottoms stream is 1.0% by weight or less.

### Example 18

The process of Example 17 is repeated with additional steps. In this example, the concentration of formaldehyde in the distillation bottoms stream is from 0.1 to 1.0% by weight.

### Example 19

The process of Example 18 is repeated with additional steps. In this example, said treated product stream flows through a filtration system to remove trace metals prior to entering the continuous distillation column.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also the individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±8%, or ±10%, of the numerical value(s) being modified. In addition, the phrase “about ‘x’ to ‘y’” includes “about ‘x’ to about ‘y’”.

While the illustrative embodiments of the invention have been described with particularity, it will be understood that the invention is capable of other and different embodiments and that various other modifications will be apparent to and may be readily made by those skilled in the art without departing from the spirit and scope of the invention. Accordingly, it is not intended that the scope of the claims hereof be limited to the examples and descriptions set forth herein but rather that the claims be construed as encompassing all the features of patentable novelty which reside in the present disclosure, including all features which would be treated as equivalents thereof by those skilled in the art to which the invention pertains.

What is claimed:

1. An improved method for removing formaldehyde from a crude butynediol product stream comprising the steps of:
(a) providing a crude butynediol stream containing butynediol and formaldehyde;
(b) increasing the pH of the crude butynediol stream and forming a treated product stream;
(c) flowing said treated product stream into the inlet of a continuous distillation column;
(d) recovering a concentrated formaldehyde stream from the overhead stream of the distillation column; and
(e) recovering a concentrated butynediol stream from the bottoms stream of the distillation column.

2. The method of claim 1 wherein of the pH of the treated product stream is raised to a level that limits the reaction of butynediol and formaldehyde to form acetal complex and decreasing the solubility of trace metals found in the treated product stream, wherein the trace metals precipitate in the treated product stream.

3. The method of claim 1 wherein the pH level of the treated product stream is increased to a range of 5.5 to 10.

4. The method of claim 1 wherein the pH level of the treated product stream is increased through the addition of a pH control agent.

5. The method of claim 4 wherein the pH control agent is an aqueous sodium hydroxide solution.

6. The method of claim 1 wherein the pH of the treated product stream is increased though the use of ion exchange resins.

7. The method of claim 1 wherein the concentration of formaldehyde in the distillation bottoms stream is 1.0% by weight or less.

8. The method of claim 7 wherein the concentration of formaldehyde in the distillation bottoms stream is from 0.1 to 1.0% by weight.

9. The method of claim 1 further comprising the step of flowing said treated product stream through a filtration system to remove trace metals prior to step (c).

10. A method for reducing the steam consumption needed for a process for removing formaldehyde from a crude butynediol product stream comprising the steps of:
(a) providing a crude butynediol stream containing butynediol and formaldehyde;
(b) increasing the pH of the crude butynediol stream and forming a treated product stream;
(c) flowing said treated product stream into the inlet of a continuous distillation column;
(d) recovering a concentrated formaldehyde stream from the overhead stream of the distillation column; and
(e) recovering a concentrated butynediol stream from the bottoms stream of the distillation column.

11. The method of claim 10 wherein of the pH of the treated product stream is raised to a level that limits the reaction of butynediol and formaldehyde to form acetal complex and decreasing the solubility of trace metals found in the treated product stream, wherein the trace metals precipitate in the treated product stream.

12. The method of claim 10 wherein the pH level of the treated product stream is increased to a range of 5.5 to 10.

13. The method of claim 10 wherein the pH level of the treated product stream is increased through the addition of a pH control agent.

14. The method of claim 13 wherein the pH control agent is an aqueous sodium hydroxide solution.

15. The method of claim 10 wherein the pH of the treated product stream is increased though the use of ion exchange resins.

16. The method of claim 10 wherein the concentration of formaldehyde in the distillation bottoms stream is 1.0% by weight or less.

17. The method of claim 16 wherein the concentration of formaldehyde in the distillation bottoms stream is from 0.1 to 1.0% by weight.

18. The method of claim 10 further comprising the step of flowing said treated product stream through a filtration system to remove trace metals prior to step (c).
